(19) United States
(12) Patent Application Publication
(10) Pub. No.: US 2007/0270955 A1
(43) Pub. Date: Nov. 22, 2007

(54) Arthoscopic arthroplasty procedure for the repair or reconstruction of arthritic joints

(76) Inventor: James C.Y. Chow, Mount Vernon, IL (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/401,500

(22) Filed: Apr. 10, 2006

(51) Int. Cl. A61F 2/44

(52) U.S. Cl. 623/17.11

(57) ABSTRACT

An arthoscopic arthroplasty procedure for the repair or reconstruction of an arthritic joint (J) in a person's hand or foot. A first opening hole (10) is made in a joint membrane (11), as is a second opening (14) for insertion of an arthoscope (16) into the joint to view the joint. A probe (12) is inserted into the joint to scrape away and remove arthritic material and diseased bone. A canula (18) is then inserted into the one opening for graft material, in the form of compressed pellets (22, 24), to be delivered to the joint. As the pellets exit the canula, they expand to a size which prevents their subsequent withdrawal from the joint through either hole. With the arthoscope, the surgeon is able to see that all the arthritic and diseased material is first removed from the joint, that the appropriate amount of graft material is then inserted into the joint to provide the cushioning required between the bones.

### CROSS REFERENCE TO RELATED APPLICATIONS

None

### STATEMENT REGARDING FEDERALLY SPONSORED RESEARCH OR DEVELOPMENT

N/A

### BACKGROUND OF THE INVENTION

This invention is related to the repair or reconstruction of arthritic small joints in a person's hands or feet, and particularly through use of arthroscopy and arthroscopic surgical procedures.

Arthritis is a disease which causes degenerative changes in a person over a period of time, and which progresses to joint stiffness and/or deformities. A person having these deformities and suffering from the accompanying pain will be able to perform only limited activities and have only limited motion. If the affected joints are in a person's hands or feet, the patient will be unable to perform such normal daily functions as walking, or making a firm grip.

Treating arthritis in its early stages can be done non-surgically. For example, the patient may be given anti-inflammatory medications such as aspirins or Cox-2 inhibitors. Other non-surgical treatments include the use of splints, physical therapy, steroid injections, etc. However, if the joint disease progresses to the point of bone rubbing against bone, the patient will experience severe pain and can be disabled. At this point, non-surgical means are no longer effective, and surgical procedures are required.

There are currently a number of surgical procedures that have been described in the relevant literature. For example, an arthroplasty procedure for arthritis is to remove the arthritic tissue and diseased bones and replace them with tissues or implants, to separate the bones and maintain normal joint function. The tissue implanted between the bones may be an autograft, which is a tissue from the patient himself; or an allograft, which is composed from cadaver tissues. An implant would be a prosthesis that separates the bones.

Further, numerous procedures have been described for use with different joints; for example, the basal joint in a person's hand, which is also called the CMC joint. Regardless of the procedure is being used, and more than twenty (20) procedures are described in the literature, the surgery requires opening the joint capsule and then closing it after the procedure is completed. The joint capsule must then heal to allow the tissue graft or implant to stay in place. There is a possibility of the graft or prosthesis becoming displaced before the healing occurs, and if this happens, then the surgery has failed and the procedure will have to be repeated.

### BRIEF SUMMARY OF THE INVENTION

The present invention is directed to an arthoscope arthroplasty procedure for the repair or reconstruction of an arthritic joint in a person's hand or foot. First and second openings are made in the joint capsule between the ends of adjacent bones. An arthroscope is inserted in one of the openings to observe the joint. A surgical instrument such as a scraping tool is then inserted into the joint through the other opening to clean out the joint by scraping away and removing arthritic joint and bone tissue from the joint. After the tool is removed, another tool such as a cannula is inserted into the opening for graft material, in the form of compressed pellets of tissue or a prosthetic material, to be delivered to the joint. As the pellets exit the cannula, they expand to a size which prevents their subsequent withdrawal from the joint through either opening.

With the arthoscope, the surgeon is first able to see that all arthritic tissue is removed from the joint, and then that an appropriate amount of graft material is inserted into the joint capsule so to provide the requisite cushioning between the bones. Once the joint is properly reconstructed and healed, the patient is able to use their toe or finger free from pain.

Other objects and features will be in part apparent and in part pointed out hereinafter.

### BRIEF DESCRIPTION OF THE SEVERAL VIEWS OF THE DRAWINGS

The objects of the invention are achieved as set forth in the illustrative embodiments shown in the drawings which form a part of the specification.

FIG. 1 illustrates a prior art joint replacement procedure;

FIG. 2 illustrates a first step in the process of the present invention;

FIG. 3 illustrates a second step in the process; and,

FIGS. 4A and 4B illustrate the insertion of different graft materials into a joint during the surgery.

Corresponding reference characters indicate corresponding parts throughout the several views of the drawings.

### DETAILED DESCRIPTION OF INVENTION

The following detailed description illustrates the invention by way of example and not by way of limitation. This description will clearly enable one skilled in the art to make and use the invention, and describes several embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

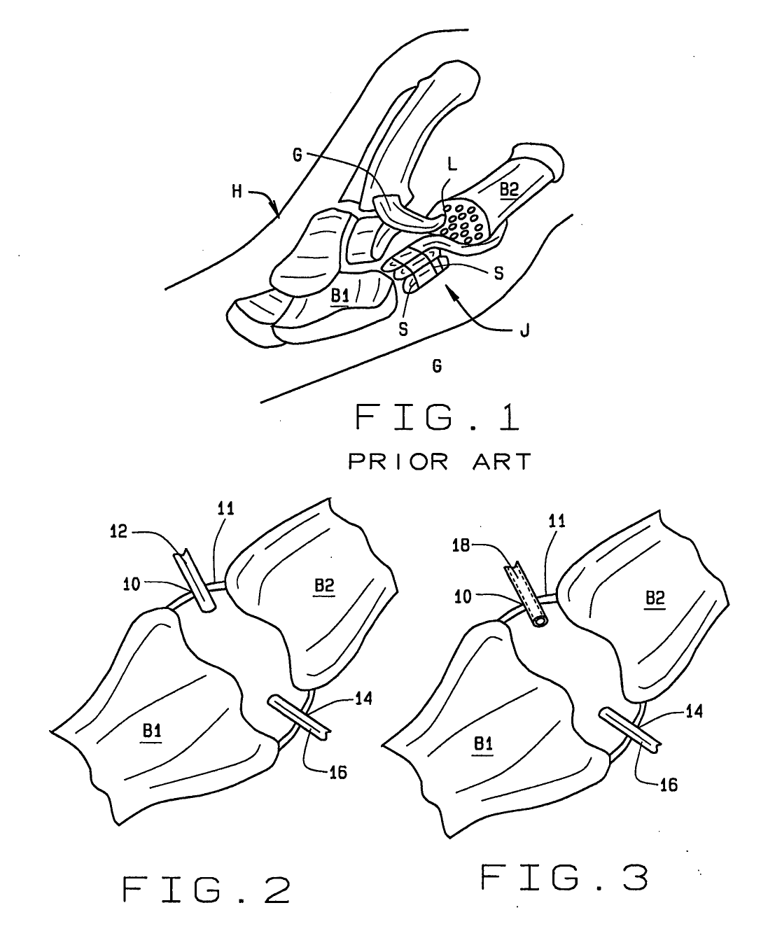

Referring to the drawings, FIG. 1 illustrates a prior art procedure in which the joint J between two adjacent bones B1, B2 in a person's hand H is replaced using a graft G. In FIG. 1, a piece of tendon from an adjacent finger is used to affect the joint replacement. A hole L is bored through bone B2, and the section of tendon routed through the hole. The outer end of the section is then formed into folds which are held together using sutures S. The completed graft is situated between adjacent ends of bones B1, B2 to affect the necessary cushioning in the joint between the bones. Some of the difficulties and problems with this procedure are previously discussed.

An arthroscope surgical procedure of the present invention provides for reconstruction or repair of the joint without having these difficulties and problems. In accordance with the invention, and as shown in FIG. 2, an opening 10 is made in the membrane 11 defining the capsule in which the joint tissue is housed. A surgical device or instrument such as tool 12 is inserted through this opening into the joint. Another opening 14 is also made in membrane 11, and an arthoscope 16 is inserted into the joint through this opening. As is known in the surgical arts, an arthoscope is an optical device or probe which allows a surgeon to readily view a surgical site within the patient's body. Typically, the arthoscope is first inserted into the joint through opening 14, and then tool 12 is inserted into the joint through opening 10.

In the next step of the procedure, tool 12 is used to scrape away the arthritic joint tissue and diseased portions of bones B1, B2. As this is done, the accumulating tissue and bone is drawn off from the joint, using suction, for example. Tool 12 may, for example, be a shaver, a burr, or clamps, or other tools appropriate for removing portions of the bone. Further, it sometimes occurs that the entire bone must be removed because of the extent of the disease. Regardless, before proceeding to the next step, it is necessary that joint J be cleansed of all diseased material.

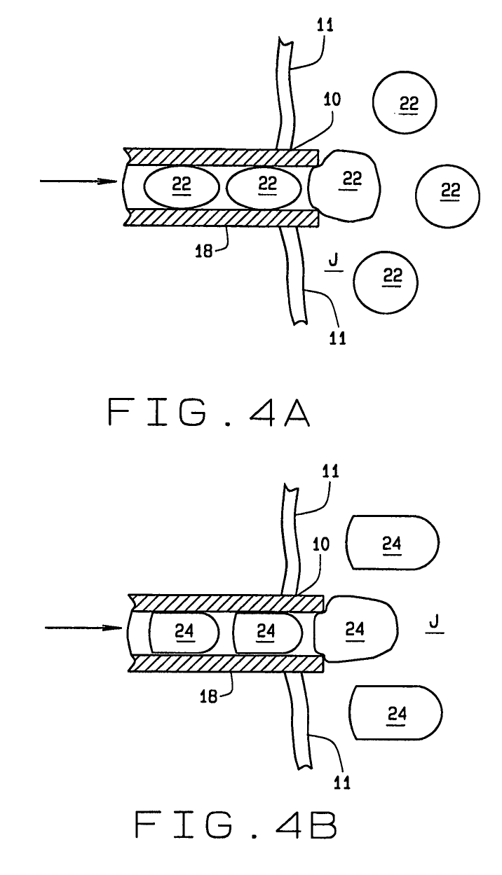

Next, and as shown in FIG. 3, tool 12 is removed and a tube or cannula 18 is inserted through opening 10 in joint membrane 11. Once in place, the tube is used to insert graft material into the joint. The graft material can be either tissue taken from the patient's own body, tissue from another living person (a donor) or from a cadaver, tissue from an animal, or an artificial, prosthetic material. As particularly shown in FIGS. 4A and 4B, the graft material is formed into pellets 22 (FIG. 4A) or 24 (FIG. 4B). In FIG. 4A, the pellets 22 have a round shape; while in FIG. 4B, the pellets 24 have a bullet shape. Those skilled in the art will appreciate that other pellets shapes can be used, and that the particular shape of pellet used depends, for example, upon the size and shape of the joint being repaired or reconstructed by the surgery.

Importantly, regardless of the shape of the pellet, the pellets, which are malleable, are compressed as they are inserted into the outer end of tube 18. They are then pushed through the tube as shown in FIGS. 4A and 4B. The force used to push them can be either human or mechanical. In the one instance, the surgeon or his assistant pushes the pellets through the tube using an appropriate tool. In the other instance, the outer end of tube 18 is connected to a mechanical or pneumatic device which imparts sufficient force or pressure on the pellets to push them through the tube. As shown in FIGS. 4A and 4B, as a pellet 22 or 24 reaches the inner end of tube 18, it expands in size as it exits the tube. The amount of expansion is such that the size of the pellet is now greater than the diameter of either opening 10 or 14 and the pellet cannot escape from the joint through either opening, but rather is retained within the joint J capsule.

This graft material delivery procedure is important for a number of reasons. As previously noted, a major problem with current surgical procedures is having to form the graft material into a suitable shape once it is positioned within the joint. A second problem is insuring the graft material stays in the joint and does not somehow escape from the joint. Using the pellets 22 or 24, and forcing the pellets through an opening smaller than their expanded size, solves both problems. First of all, the individual pellets are much easier to handle than a length of graft material such as the graft material G in FIG. 1. Second, the pellets are confined within the joint capsule and cannot be escape through either opening in the joint membrane. In the procedure of FIG. 1, for example, if the sutures S break or disintegrate over time, the graft material could work its way back out of the joint through hole L, necessitating another surgery to repair the problem.

As pellets are inserted into joint J, they tend to fill the space defined by joint membrane 11. Accordingly, once a sufficient number of pellets have been injected into the joint, they provide an adequate amount of cushioning between bones B1, B2 so that the bones no longer scrape together when the patient moves his hand or foot and cause the patient pain. Once all of the pellets have been injected into the joint, both cannula 18 and arthoscope 16 are withdrawn from joint capsule 11 through their respective openings, and the surgeon now closes these openings in any convenient manner. Importantly for the patient, no stitches inside his or her body need to be made, particularly any stitches to keep the graft material in place. Also, no cast is required for the hand or foot. Rather, the patient will start to regain full use of his hand or foot relatively quickly and will regain full use of the hand or foot significantly faster than with previous arthroplastic procedures.

Finally, although not shown in the drawings, those skilled in the art will understand that the surgeon can use reposition the arthroscope from opening 14 to opening 10 at various times during the procedure, as well reposition his tools from opening 10 to opening 14 for performing the above described steps of the procedure and for viewing the joint as the procedure is being carried out.

In view of the above, it will be seen that the several objects and advantages of the present invention have been achieved and other advantageous results have been obtained.

Having thus described the invention, what is claimed and desired to be secured by Letters Patent is:

1. An arthroplastic procedure for replacing an arthritic joint between two bones adjacent the joint comprising:
forming an opening in a membrane of the joint; and,
delivering graft material through the opening into the joint, the graft material expanding in size as it enters the joint to a size which prevents it from subsequently being removed from the joint, the graft material providing a cushion between adjacent ends of the bones to enable a patient to thereafter use the joint substantially free from pain.

2. The procedure of claim 1 in which the graft material is formed into pellets which are compressed in size for insertion through the opening into the joint and which expand in size as they enter the joint.

3. The procedure of claim 2 further including inserting a hollow surgical tool into the opening in the membrane and delivering the graft material to the joint through the tool.

4. The procedure of claim 1 in which the graft material is tissue taken from the patient's own body.

5. The procedure of claim 1 in which the graft material is tissue taken from another living person.

6. The procedure of claim 1 in which the graft material is tissue taken from a cadaver.

7. The procedure of claim 1 in which the graft material is tissue taken from an animal.

8. The procedure of claim 1 in which the graft material is an artificial, prosthetic material.

9. The procedure of claim 1 further including forming a second opening in the joint membrane.

10. The procedure of claim 8 further including inserting an arthoscope through the second opening for observing the joint and delivery of the graft material to the joint.

11. The procedure of claim 1 further including first inserting a surgical tool through the opening for removing arthritic tissue and diseased bone from the joint.

12. An arthoscopic arthroplasty procedure for the repair or reconstruction of an arthritic joint between two bones adjacent the joint comprising:
forming first and second openings in a joint membrane;
inserting a hollow, surgical device into one of the openings and an arthoscope through the other opening for observing the joint; and,
delivering graft material through the surgical device into the joint, the graft material expanding in size as it enters the joint to a size which prevents it from subsequently being removed from the joint, the graft material providing a cushion between adjacent ends of the bones to enable a patient to thereafter use the joint substantially free from pain.

13. The procedure of claim 12 in which the graft material is formed into pellets which are compressed in size for insertion through the device into the joint and which expand in size as they enter the joint.

14. The procedure of claim 12 in which the graft material comprises one of tissue taken from the patient's own body, tissue taken from another living person, tissue taken from a cadaver, or tissue taken from an animal.

15. The procedure of claim 12 in which the graft material comprises an artificial, prosthetic material.

16. The procedure of claim 12 further including first inserting a surgical tool through the first said opening and using the tool to remove arthritic tissue from the joint.

17. An arthoscopic arthroplasty procedure for the repair or reconstruction of an arthritic joint between two bones adjacent the joint comprising comprising:
forming first and second openings in a joint membrane;
inserting an arthoscope through one of the openings for observing the joint;
inserting a surgical tool through the other said opening, and using the tool to remove arthritic tissue and diseased bone material from the joint; and,
removing the tool from the other said opening, inserting a hollow, surgical device thereinto, and delivering graft material through the surgical device into the joint, the graft material expanding in size as it enters the joint to a size which prevents it from subsequently being removable from the joint, the graft material providing a cushion between adjacent ends of the bones to enable a patient to thereafter use the joint substantially free from pain.

18. The procedure of claim 17 in which the graft material is formed into pellets which are compressed in size for insertion through the device into the joint and which expand in size as they enter the joint.

19. The procedure of claim 17 in which the graft material comprises one of tissue taken from the patient's own body, tissue taken from another living person, tissue taken from a cadaver, or tissue taken from an animal.

20. The procedure of claim 17 in which the graft material comprises an artificial, prosthetic material.
